SEQUENCE LISTING (1 sequence)

SEQ ID NO: 1 - DNA, 7399 bp, coxsackievirus B3
  source 1..7399: mol_type = unassigned DNA
ttaaaacagcctgtgggttgatcccacccacagggcccattgggcgctagcactctggtatcacggtacctttgtgcgcctgttttataccccctctcccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaattccaactgcggagcacacaccctcaagccagagggcagtgtgtcgtaacgggcaactctgcagcggaaccgactactttgggtgtccgtgtttcattttattcctatactggctgcttatggtgacaattgagagattgttaccatatagctattggattggccatccggtgactaatagagctattatatatccctttgttgggtttataccacttagcttgaaagaggttaaaacattacaattcattgttaagttgaatacagcaaaatgggagctcaagtatcaacgcaaaagactggggcacatgagaccaggctgaatgctagcggcaattccatcattcactacacaaatattaattattacaaggatgccgcatccaactcagccaatcggcaggatttcactcaagacccgggcaagttcacagaaccagtaaaagatatcatgattaaatcactaccagctctcaactcccccacagtagaggagtgcggatacagtgacagggcgagatcaatcacattaggtaactccaccataacgactcaggaatgcgccaacgtggtggtgggctatggagtatggccagattatctaaaggatagtgaggcaacagcagaggaccaaccgacccaaccagacgttgccacatgtaggttctatacccttgactctgtgcaatggcagaaaacctcaccagggtggtggtggaagctgcccgatgctttgtcgaacttaggactgtttgggcagaacatgcagtaccactacttaggccgaactgggtataccgtacatgtgcagtgcaatgcatctaagttccaccaaggatgcttgctagtagtgtgtgtaccggaagctgagatgggttgcgcaacgctagacaacaccccatccagtgcagaattgctggggggcgatagcgcaaaagagtttgcggacaaaccggtcgcatccgggtccaacaagttggtacagagggtggtgtataatgcaggcatgggggtgggtgttggaaacctcaccattttcccccaccaatggatcaacctacgcaccaataatagtgctacaattgtgatgccatacaccaacagtgtacctatggataacatgtttaggcataacaacgtcaccctaatggttatcccatttgtaccgctagattactgccctgggtccaccacgtacgtcccaattacggtcacgatagccccaatgtgtgccgagtacaatgggttacgtttagcagggcaccagggcttaccaaccatgaatactccggggagctgtcaatttctgacatcagacgacttccaatcaccatccgccatgccgcaatatgacgtcacaccagagatgaggatacctggtgaggtgaaaaacttgatggaaatagctgaggttgactcagttgtcccagtccaaaatgttggagagaaggtcaactctatggaagcataccagatacctgtgagatccaacgaaggatctggaacgcaagtattcggctttccactgcaaccagggtactcgagtgtttttagtcggacgctcctaggagagatcttgaactattatacacattggtcaggcagcataaagcttacgtttatgttctgtggttcggccatggctactggaaaattccttttggcatactcaccaccaggtgctggagctcctacaaaaagggttgatgccatgcttggtactcatgtaatttgggacgtggggctacaatcaagttgcgtgctgtgtataccctggataagccaaacacactaccggtatgttgcttcagatgagtataccgcagggggttttattacgtgctggtatcaaacaaacatagtggtcccagcggatgcccaaagctcctgttacatcatgtgtttcgtgtcagcatgcaatgacttctctgtcaggctattgaaggacactcctttcatttcgcagcaaaactttttccagggcccagtggaagacgcgataacagccgctatagggagagttgcggataccgtgggtacagggccaaccaactcagaagctataccagcactcactgctgctgagacgggtcacacgtcacaagtagtgccgggtgacactatgcagacacgccacgttaagaactaccattcaaggtccgagtcaaccatagagaacttcctatgtaggtcagcatgcgtgtactttacggagtatgaaaactcaggtgccaagcggtatgctgaatgggtattaacaccacgacaagcagcacaacttaggagaaagctagaattctttacctacgtccggttcgacctggagctgacgtttgtcataacaagtactcaacagccctcaaccacacagaaccaagatgcacagatcctaacacaccaaattatgtatgtaccaccaggtggacctgtaccagataaagttgattcatacgtgtggcaaacatctacgaatcccagtgtgttttggaccgagggaaacgccccgccgcgcatgtccataccgtttttgagcattggcaacgcctattcaaatttctatgacggatggtctgaattttccaggaacggagtttacggcatcaacacgctaaacaacatgggcacgctatatgcaagacatgtcaacgctggaagcacgggtccaataaaaagcaccattagaatctacttcaaaccgaagcatgtcaaagcgtggatacctagaccacctagactctgccaatacgagaaggcaaagaacgtgaacttccaacccagcggagttaccactactaggcaaagcatcactacaatgacaaatacgggcgcatttggacaacaatcaggggcagtgtatgtggggaactacagggtggtaaatagacatctagctaccagtgctgactggcaaaactgtgtgtgggaaagttacaacagagacctcttagtgagcacgaccacagcacatggatgtgatattatagccagatgtcagtgcacaacgggagtgtacttttgtgcgtccaaaaacaagcactacccaatttcgtttgaaggaccaggtctagtagaggtccaagagagtgaatactaccccaggagataccaatcccatgtgcttttagcagctggattttccgaaccaggtgactgtggcggtatcctaaggtgtgagcatggtgtcattggcattgtgaccatggggggtgaaggcgtggtcggctttgcagacatccgtgatctcctgtggctggaagatgatgcaatggaacagggagtgaaggactatgtggaacagcttggaaatgcattcggctccggctttactaaccaaatatgtgagcaagtcaacctcctgaaagaatcactagtgggtcaagactccatcttagagaaatctctaaaagccttagttaagataatatcagccttagtaattgtggtgaggaaccacgatgacctgatcactgtgactgccacactagcccttatcggttgtacctcgtccccgtggcggtggctcaaacagaaggtgtcacaatattacggaatccctatggctgaacgccaaaacaatagctggcttaagaaatttactgaaatgacgaatgcttgcaagggtatggaatggatagctgtcaaaattcagaaattcattgaatggctcaaagtaaaaattttgccagaggtcagggaaaaacacgagttcctgaacagacttaaacaactccccttattagaaagtcagatcgccacaatcgagcagagcgcgccatcccaaagtgaccaggaacaattattttccaatgtccaatactttgcccactattgcagaaagtacgctcccctctacgcggctgaagcaaagagggtgttctcccttgagaagaagatgagcaattacatacagttcaagtccaaatgccgtattgaacctgtatgtttgctcctgcacgggagccctggtgccggcaagtcggtggcaacaaacttaattggaaggtcgcttgctgagaaactcaacagctcagtgtactcactaccgccagacccagatcacttcgacggatacaaacagcaggccgtggtgattatggacgatctatgccagaatcctgatgggaaagacgtctccttgttctgccaaatggtttccagtgtagattttgtaccacccatggctgccctagaagagaaaggcattctgttcacctcaccgtttgtcttggcatcgaccaatgcaggatctattaatgctccaaccgtgtcagatagcagagccttggcaaggagatttcactttgacatgaacatcgaggttatttccatgtacagtcagaatggcaagataaacatgcccatgtcagtcaagacttgtgacgatgagtgttgcccggtcaattttaaaaagtgctgccctcttgtgtgtgggaaggctatacaattcattgatagaagaacacaggtcagatactctctagacatgctagtcaccgagatgtttagggagtacaatcatagacatagcgtggggaccacgcttgaggcactgttccagggaccaccagtatacagagagatcaaaattagcgttgcaccagagacaccaccaccgctcgccattgcggacctgctcaaatcggtagacagtgaggctgtgagggagtactgcaaagaaaaaggatggttggttcctgagatcaactccaccctccaaattgagaaacatgtcagtcgggctttcatttgcttacaggcattgaccacatttgtgtcagtggctggaatcatatatataatatataagctctttgcgggttttcaaggtgcttatacaggagtgcccaaccagaagcccagagtgcctaccctgaggcaagcaaaagtgcaaggccctgcctttgagttcgccgtcgcaatgatgaaaaggaactcaagcacggtgaaaactgaatatggcgagtttaccatgctgggcatctatgacaggtgggccgttttgccacgccacgccaaacctgggccaaccatcttgatgaatgatcaagaggttggtgtgctagatgccaaggagctagtagacaaggacggcaccaacttagaactgacactactcaaattgaaccggaatgagaagttcagagacatcagaggcttcttagccaaggaggaagtggaggttaatgaggcagtgctagcaattaacaccagcaagtttcccaacatgtacattccagtaggacaggtcacagaatacggcttcctaaacctaggtggcacacccaccaagagaatgcttatgtacaacttccccacaagagcaggccagtgtggtggagtgctcatgtccaccggcaaggtactgggtatccatgttggtggaaatggccatcagggcttctcagcagcactcctcaaacactacttcaatgatgagcaaggtgaaatagaatttattgagagctcaaaggacgccgggtttccagtcatcaacacaccaagtaaaacaaagttggagcctagtgttttccaccaggtctttgaggggaacaaagaaccagcagtactcaggagtggggatccacgtctcaaggccaattttgaagaggctatattttccaagtatataggaaatgtcaacacacacgtggatgagtacatgctggaagcagtggaccactacgcaggccaactagccaccctagatatcagcactgaaccaatgaaactggaggacgcagtgtacggtaccgagggtcttgaggcgcttgatctaacaacgagtgccggttacccatatgttgcactgggtatcaagaagagggacatcctctctaagaagactaaggacctaacaaagttaaaggaatgtatggacaagtatggcctgaacctaccaatggtgacttatgtaaaagatgagctcaggtccatagagaaggtagcgaaaggaaagtctaggctgattgaggcgtccagtttgaatgattcagtggcgatgagacagacatttggtaatctgtacaaaactttccacctaaacccaggggttgtgactggtagtgctgttgggtgtgacccagacctcttttggagcaagataccagtgatgttagatggacatctcatagcatttgattactctgggtacgatgctagcttaagccctgtctggtttgcttgcctaaaaatgttacttgagaagcttggatacacgcacaaagagacaaactacattgactacttgtgcaactcccatcacctgtacagggataaacattactttgtgaggggtggcatgccctcgggatgttctggtaccagtattttcaactcaatgattaacaatatcataattaggacactaatgctaaaagtgtacaaagggattgacttggaccaattcaggatgatcgcatatggtgatgatgtgatcgcatcgtacccatggcctatagatgcatctttactcgctgaagctggtaagggttacgggctgatcatgacaccagcagataagggagagtgctttaacgaagttacctggaccaacgccactttcctaaagaggtattttagagcagatgaacagtaccccttcctggtgcatcctgttatgcccatgaaagacatacacgaatcaattagatggaccaaggatccaaagaacacccaagatcacgtgcgctcactgtgtctattagcttggcataacggggagcacgaatatgaggagttcatccgtaaaattagaagcgtcccagtcggacgttgtttgaccctccccgcgttttcaactctacgcaggaagtggttggactccttttagattagagacaatttgaaataatttagattggcttaaccctactgtgctaaccgaaccagataacggtacagtaggggtaaattctccgcattcggtgcg

(19) United States
(12) Patent Application Publication
(10) Pub. No.: US 2024/0261386 A1
(43) Pub. Date: Aug. 8, 2024

(54) Coxsackievirus 83 Vaccine

(71) Applicant: KING FAISAL UNIVERSITY, AL-AHSA (SA)

(72) Inventors: JAWHAR GHARBI, AL-AHSA (SA); MOHAMMED A. ALMALKI, AL-AHSA (SA); AMEERA ALYAMI, AL-AHSA (SA); MANEL BEN M'HADHEB, AL-AHSA (SA)

(21) Appl. No.: 18/165,762

(22) Filed: Feb. 7, 2023

(51) Int. Cl. A61K 39/125; A61K 9/48; A61P 31/14; C07K 14/005

(52) U.S. Cl. CPC A61K 39/125; A61K 9/485; A61P 31/14; C07K 14/005; A61K 2039/5254; A61K 2039/54; C12N 2770/32321; C12N 2770/32322; C12N 2770/32334; C12N 2770/32371

(57) ABSTRACT

The Coxsackievirus B3 (CVB3) vaccine including a mutant strain of Coxsackievirus B3 (CVB3) (SEQ ID NO: 1) has specific double mutations introduced in the Internal Ribosome Entry Segment (IRES) region of the wild type Coxsackievirus B3 (CVB3) genome in the nucleotide positions 473 (in which uracil is substituted for cytosine) and 475 (in which cytosine is substituted for uracil). The resulting double mutant (SEQ ID NO: 1) demonstrates a significant decrease in its replicative capacity and a drastic decrease in its translation efficiency compared to the wild-type Coxsackievirus B3 (CVB3) strain.

### BACKGROUND

### Sequence Listing Xml

The instant application contains a Sequence Listing XML, which has been submitted in XML format via the USPTO's Patent Center and is hereby incorporated by reference in its entirety. The XML copy, created on Jan. 30, 2023, is named 32087_70_U_SEQUENCE.xml and is 9,000 bytes in size.

### 1. FIELD

The disclosure of the present patent application relates to vaccines against Coxsackievirus B3 (CVB3) infection, and particularly to a Coxsackievirus B3 vaccine including a genetically modified strain of CVB3 that exhibits an attenuated virulence when compared to the wild-type CVB3 strain.

### 2. DESCRIPTION OF THE RELATED ART

Viruses of the genus Enterovirus affect millions of people worldwide each year, and are often found in the respiratory secretions (e.g., saliva, sputum, or nasal mucus) and stool of an infected person. Enterovirus infects the gut, thus the derivation of their name from the root “enteric”. Historically, poliomyelitis was the most significant disease caused by an enterovirus, that is, poliovirus. There are 62 non-polio Enteroviruses that can cause disease in humans: 23 Coxsackie A viruses, 6 Coxsackie B viruses, 28 echoviruses, and 5 other enteroviruses. Polioviruses, as well as Coxsackie viruses and echoviruses, are spread through the fecal-oral route. Infection can result in a wide variety of symptoms, including mild respiratory illness (common cold), hand, foot and mouth disease, acute hemorrhagic conjunctivitis, aseptic meningitis, myocarditis, severe neonatal sepsis-like disease, and acute flaccid paralysis.

Enterovirus represents a genus of a large and diverse group of small RNA viruses characterized by a single positive-strand genomic RNA. All enteroviruses contain a genome of approximately 7,500 bases and are known to have a high mutation rate due to low-fidelity replication and frequent recombination. After infection of the host cell, the genome is translated in a cap-independent manner into a single polyprotein, which is subsequently processed by virus-encoded proteases into the structural capsid proteins and the nonstructural proteins, which are mainly involved in the replication of the virus.

The enteroviruses are associated with several human and mammalian diseases. Serologic studies have distinguished 66 human serotypes on the basis of antibody neutralization tests. Additional antigenic variants have been defined within several of the serotypes on the basis of reduced or nonreciprocal cross-neutralization between variant strains. On the basis of their pathogenesis in humans and animals, enteroviruses were originally classified into four groups, namely, polioviruses, Coxsackie A viruses (CA), Coxsackie B viruses (CB), and echoviruses, but it was quickly realized that there were significant overlaps in the biological properties of viruses in the different groups.

Coxsackievirus B3 (CVB3) is a typical human enterovirus of the family Picornaviridae. The CVB3 genome is a single molecule of positive sense RNA that encodes a 2,185 amino acid polyprotein. The single long open reading frame is flanked by a 5′ non-translated region (5′ NTR), 742 nucleotides long, and a much shorter 3′ NTR, which terminates in a polyadenylate tract. Like the polioviruses (PVs), CVB3 shuts off host cell protein translation in infected HeLa cells. The near atomic structure of the CVB3 virion has been solved, demonstrating that the CVB3 capsid shares a similar capsid structure with genetically-related entero- and rhinoviruses. However, no satisfactory preventive measure of treatment for CVB3 infection is known.

Thus, a vaccine against CVB3 solving the aforementioned problems is desired.

### SUMMARY

The Coxsackievirus B3 vaccine is directed to a vaccine including a mutant strain of Coxsackievirus B3 (CVB3) (SEQ ID NO: 1) that includes specific double mutations introduced in the Internal Ribosome Entry Segment (IRES) region of the wild-type Coxsackievirus B3 (CVB3) genome in the nucleotide positions nt. 473 (in which uracil is substituted for cytosine) and nt. 475 (in which cytosine is substituted for uracil). The resulting double mutant (SEQ ID NO: 1) demonstrates a significant decrease in the virus replicative capacity and a drastic decrease in its translation efficiency compared to the wild-type Coxsackievirus B3 (CVB3) strain. When tested in a murine model, the double mutant (SEQ ID NO: 1) exhibits an important attenuated virulence phenotype in comparison with the wild-type and other produced mutants.

The attenuated virus or double mutant (SEQ ID NO: 1) is easy to produce by introducing specific mutations in a well-characterized genomic region of the whole wild-type virus genome cloned in an expression vector. The attenuated virus (SEQ ID NO: 1) can be obtained by substituting a cytosine nucleotide for a uracil nucleotide at position nt473 and substituting a uracil nucleotide for a cytosine nucleotide at position nt. 475 in the genome of the wild-type Coxsackievirus B3, by site-directed mutagenesis in the domain V of the internal ribosome entry segment (IRES). The resulting double mutant (SEQ ID NO: 1) is stable and is not susceptible to reversions after replication. The double mutant (SEQ ID NO: 1) is also viable and continues to replicate in organs and cell culture, but with a decreasing capacity. The double mutant (SEQ ID NO: 1) reveals a drastic decrease in protein translation in cell culture. The double mutant exhibits (SEQ ID NO: 1) an attenuated virulence phenotype in murine models, compared to the wild-type strain and other mutants.

The viral liquid suspension can be coated in soft or hard capsule. The capsule should not be gastro-resistant to allow primary replication of the vaccine strain (SEQ ID NO: 1) in the digestive tract (which is considered the primary site of viral replication).

These and other features of the present subject matter will become readily apparent upon further review of the following specification and drawings.

### BRIEF DESCRIPTION OF THE DRAWINGS

FIG. 1 is a schematic diagram of an expression vector used for the design of the Coxsackievirus B3 vaccine.

FIG. 2 is a chart showing the growth in a one-step cycle of wild-type and mutant CVB3 (SEQ ID NO: 1) viruses in HeLa cells.

FIG. 3A is an autoradiograph of the dried SDS-polyacrylamide gel of the RRL-based translation reactions.

FIG. 3B is a chart depicting results of densitometric quantifications of polyprotein products within the wild-type strain of CVB3 and mutant strain of CVB3 (SEQ ID NO: 1) in polyacrylamide gel.

FIG. 4 is a chart depicting evolution of anti-CVB3 neutralizing antibody after immunization by the live-attenuated double mutant (Sab-3like+C) at day 0 and challenged by CVB3 Wild-type at day 15.

FIG. 5A is a diagram depicting intraperitoneal (IP) immunization schedule of mice.

FIG. 5B is a diagram depicting oral (per os) immunization schedule of mice.

FIGS. 6A-6F are images of murine myocardium stained with hematoxylin and eosin, including (FIG. 6A) a mouse inoculated with wild-type CVB3; (FIG. 6B) a control uninfected mouse; (FIG. 6C) a mouse inoculated with Sab-3 like mutant; (FIG. 6D) a mouse inoculated with Sab-3like+C mutant (SEQ ID NO: 1); (FIG. 6E) a mouse inoculated with Sab-2like mutant; and (FIG. 6F) a mouse inoculated with Sab-1like mutant (Bars, 200 μm).

FIG. 7 is a chart depicting evolution of neutralizing IgG in sera of mice intraperitoneally (IP) and orally (per os) immunized by the live-attenuated mutant.

FIG. 8 is a chart showing levels of IgG, IgG1 and IgG2a neutralizing antibodies in the serum of intraperitoneally and orally immunized mice.

FIG. 9 is a plot comparing body weight changes observed in intraperitoneally (IP) and orally (per os) immunized mice.

FIG. 10 is a plot comparing percent of survival of intraperitoneally (IP) and orally (per os) immunized mice after a challenge with wild-type CVB3 Nancy strain in comparison to that of the negative control.

FIG. 11 is a chart of titers calculation on HeLa cell cultures of virus isolated seven days post challenge from mice organs.

FIGS. 12A-12D show myocardium tissues stained with haematoxylin and eosin, including (FIG. 12A) control challenged mice; (FIG. 12B) twice orally immunized, then challenged mice; (FIG. 12C) mice three times intraperitoneally immunized, then challenged; and (FIG. 12D) control non-challenged mice (Bars, 200 μm).

Similar reference characters denote corresponding features consistently throughout the attached drawings.

### DETAILED DESCRIPTION OF THE PREFERRED EMBODIMENTS

The following definitions are provided for the purpose of understanding the present subject matter and for construing the appended patent claims.

It should be understood that the drawings described above or below are for illustration purposes only. The drawings are not necessarily to scale, with emphasis generally being placed upon illustrating the principles of the present teachings. The drawings are not intended to limit the scope of the present teachings in any way.

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

It will be understood by those skilled in the art with respect to any chemical group containing one or more substituents that such groups are not intended to introduce any substitution or substitution patterns that are sterically impractical and/or physically non-feasible.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

“Subject”, as used herein, refers to any animal classified as a mammal, including humans, domestic and farm animals, and zoo, sports, and pet companion animals, such as household pets and other domesticated animals and including, but not limited to, cattle, sheep, ferrets, swine, horses, poultry, rabbits, goats, dogs, cats and the like.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In an embodiment, a double mutant strain of Coxsackievirus 3B (CVB3) genome (SEQ ID NO: 1) may be used as a live, attenuated vaccine for prevention of natural infection by CVB3. Attenuation of the virulent, wild-type CVB3 may be accomplished through a transcriptional mechanism. Preferred embodiments include attenuating the virus by substituting a cytosine nucleotide by a uracil nucleotide at position nt 473 in the genome of the wild type Coxsackievirus B3 (Sab3-like mutation) and substituting a uracil nucleotide by a cytosine nucleotide at position nt475 in the genome of the wild type Coxsackievirus B3 in Domain V within the internal ribosome entry segment (IRES).

In an embodiment, the two mutations, a cytosine nucleotide by a uracil nucleotide at position nt473 and a uracil nucleotide substituted by a cytosine nucleotide at position nt475 in the genome of the wild type Coxsackievirus B3, can be introduced by site-directed mutagenesis in the domain V of the IRES of the whole cDNA of a virulent wild type CVB3 strain. The cDNA of the virulent wild type CVB3 strain can be cloned in an expression plasmid vector. As described herein, an in vitro one-step cycle of the double mutant (SEQ ID NO: 1) produced by transfection of HeLa cells demonstrated a significant decrease of the yield of infectious viral particles compared to the wild type strain. Further, a drastic decrease of in vitro translation efficiency was revealed when using Rabbit reticulocyte lysates (RRL) system. In addition, based on pathogenicity testing in a mice model, it was revealed that the double mutant (SEQ ID NO: 1) exhibits an attenuated virulence phenotype compared to the wild type strain. Accordingly, the double mutant strain (SEQ ID NO: 1) can be used as a live-attenuated vaccine to prevent natural CVB 3 infection in a subject.

In an embodiment, an effective amount of a vaccine comprising attenuated Coxsackievirus 3B (CVB3) (SEQ ID NO: 1) can be administered to a subject for conferring protection and prevention against natural infections by the wild-type or virulent strain of Coxsackievirus 3B (CVB3). Persistent infections with the virulent strain of CVB3 are mainly associated with chronic diseases of the heart muscles, i.e., myocarditis and dilated cardiomyopathy. The attenuated Coxsackievirus 3B (CVB3) (SEQ ID NO: 1) can be obtained by introducing two mutations, a Sabin 3-like mutation (C/U position nucleotide 473) and an additional substitution (U/C position nucleotide 475), site-directed mutagenesis in the domain V of the IRES of the whole cDNA of a virulent wild type CVB3 strain. An effective amount of the vaccine is an amount that can elicit a protective immune response in the recipient, either by eliciting neutralizing antibodies or a cell-mediated response, or both. The vaccine can induce serotype specific immunity or neutralizing antibodies.

A method of vaccinating a subject against CVB3 infection may include administering to the subject the Coxsackievirus B3 live-attenuated vaccine in an amount effective to elicit an immune response and/or neutralizing antibody response directed against CVB3 when administered to the subject. The vaccine may be administered intraperitoneally or orally, for example. In an embodiment, the vaccine is formulated as a liquid suspension for oral administration. In an embodiment, the liquid suspension includes Phosphate-Buffered Saline. In an embodiment, the liquid suspension is provided within a capsule. The capsule is preferably not gastro-resistant, to allow primary replication of the vaccine strain in the digestive tract (which is considered the primary site of viral replication).

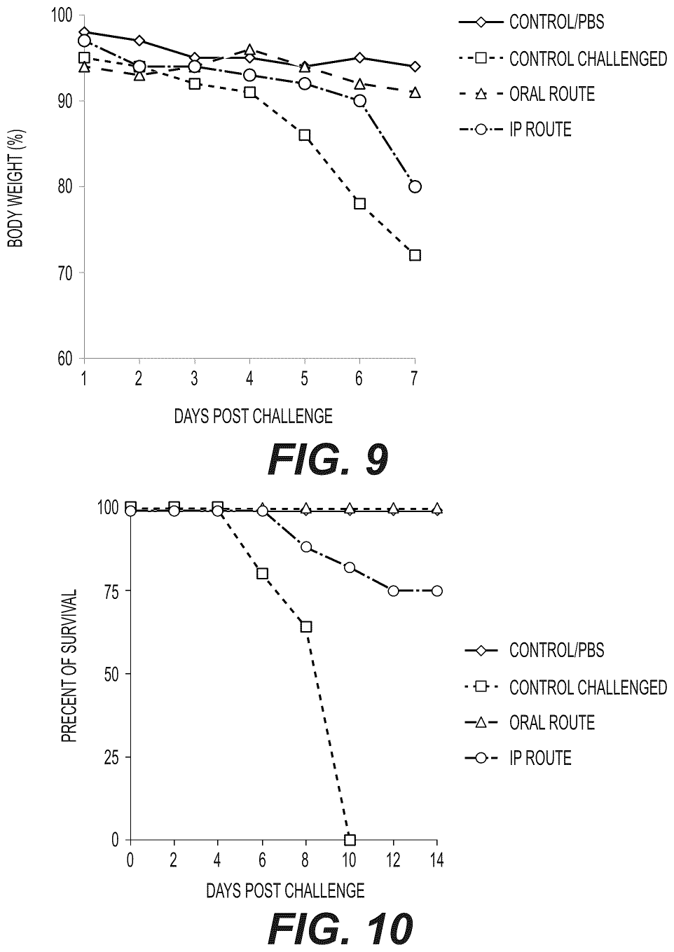

As described herein, in vivo data demonstrates attenuated virulence of vaccine strain (SEQ ID NO: 1) compared to the wild-type strain. For example, less body weight changes were observed in mice inoculated by vaccine strains (SEQ ID NO: 1) than mice inoculated by wild-type (FIG. 9). Less lethality was observed in mice inoculated by vaccine strain (SEQ ID NO: 1) compared with mice inoculated by wild-type strain (FIG. 10).

The vaccine may induce a protective immune response. The term “protective immune response” and/or “neutralizing immune response”, as used herein, is intended to mean that the vaccinated subject may resist or protect itself against an infection with the pathogenic agent against which the vaccination was done.

The present subject matter will be better understood with reference to the following examples.

### Example 1

### Expression Vector Design

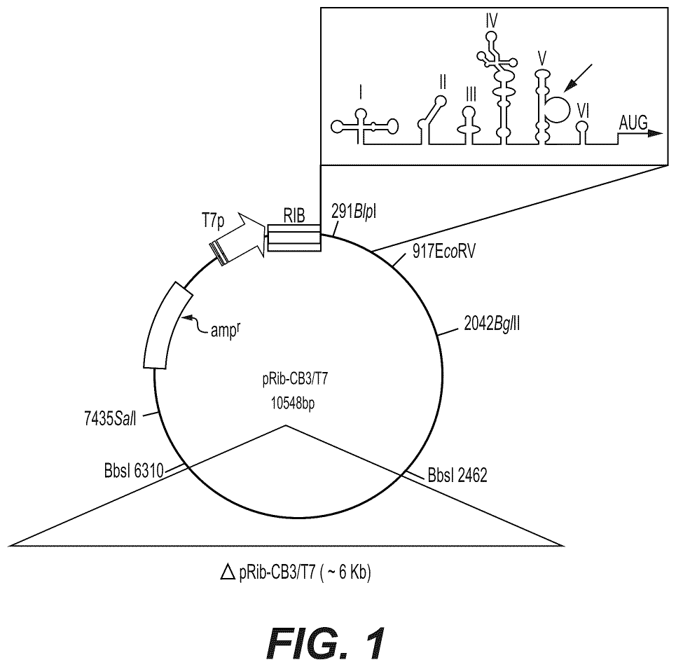

The attenuated CVB3 (SEQ ID NO: 1) was produced by introducing specific mutations in a well-characterized genomic region of the whole wild type virus genome cloned in an expression vector. FIG. 1 is a schematic overview of the expression vector used for the design of the new product. The position and secondary structure of the CVB3 IRES are represented in an extension square. The lateral bulge-loop of SL-V targeted in the study is indicated by an arrow. The double mutations (C/U position nucleotide 473 and U/C position nucleotide 475) were introduced by site-directed mutagenesis in the stem-loop V of the wild-type strain IRES of the virus (p: Plasmid; Rib: Ribozyme sequence; T7P: Transcriptase T7 Promoter; ampr: ampicillin resistance gene; A pRib: depleted plasmid; AUG: Initiation code).

### Example 2

### Decreasing of Replicative Capacity in Cell Culture of the Double Mutant

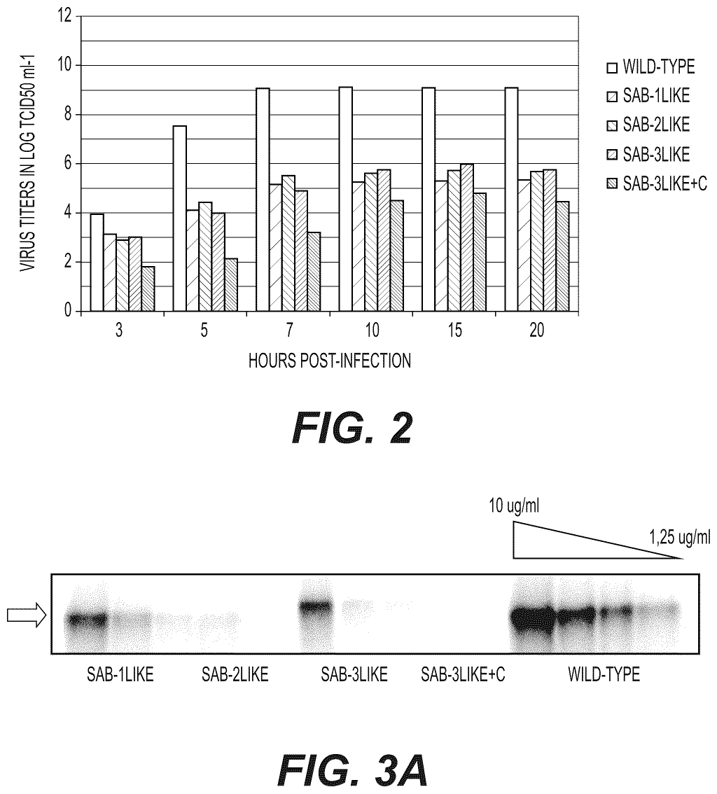

FIG. 2 depicts the growth in a one-step cycle of wild-type and mutant viruses in HeLa cells. Cells were infected at the same MOI of 10 TCID₅₀/cell. Samples were analysed for infectious virus particle production at the indicated times post-infection by determining titers on HeLa cell monolayers. Experiments were performed in duplicate. Data points are the mean of two infections.

### Example 3

### In Vitro Translation Efficiencies of CVB3 Virus Mutants

In vitro translation reactions were carried out with standard rabbit reticulocyte lysates (RRL) supplemented with HeLa cell S10 extracts prepared and treated with micrococcal nuclease. All reactions contained 50% (vol/vol) RRL (Flexi RRL system; Promega), 417 μCi of [³⁵S] methionine/ml (>1,000 Ci/mmol; Amersham), and 80 μM unlabelled amino acids (except for Leucine and Valine, which were present at 120 μM, and Methionine, which was omitted). Added MgCl₂ and KCl were optimized on the bases of the RRL used from 0.2 to 1.2 mM and 50 to 100 mM, respectively. Concentrations of uncapped mRNA were varied from 1.25 to 10 μg/ml, and that of H100 buffer (10 mM Hepes-KOH [pH 7.5], 1 mM MgCL₂, 0.1 Mm EDTA, 100 mM KCl, 7 mM β-mercaptoethanol) was varied from 33 to 13% (vol/vol) of the final reaction volume. Reactions were programmed with uncapped mRNAs at concentration of 10 μg/ml, and assay reaction mixtures were incubated for 1h at 30° C. Reactions were stopped by adding stop RNase solution and incubated for 10 min at room temperature before adding of blue solution (80% bromophenol blue, 20% β-mercaptoethanol). Translation products were analyzed by sodium dodecyl sulphate polyacrylamide gel electrophoresis by using gels containing 15% (wt/vol) polyacrylamide. Quantification of translation of the viral polyprotein before its cleavage was carried out by densitometry of autoradiograms by using NIH image software (Image J 1.34s), with multiple exposures of each radiogram to ensure that the linear response range of the film was respected.

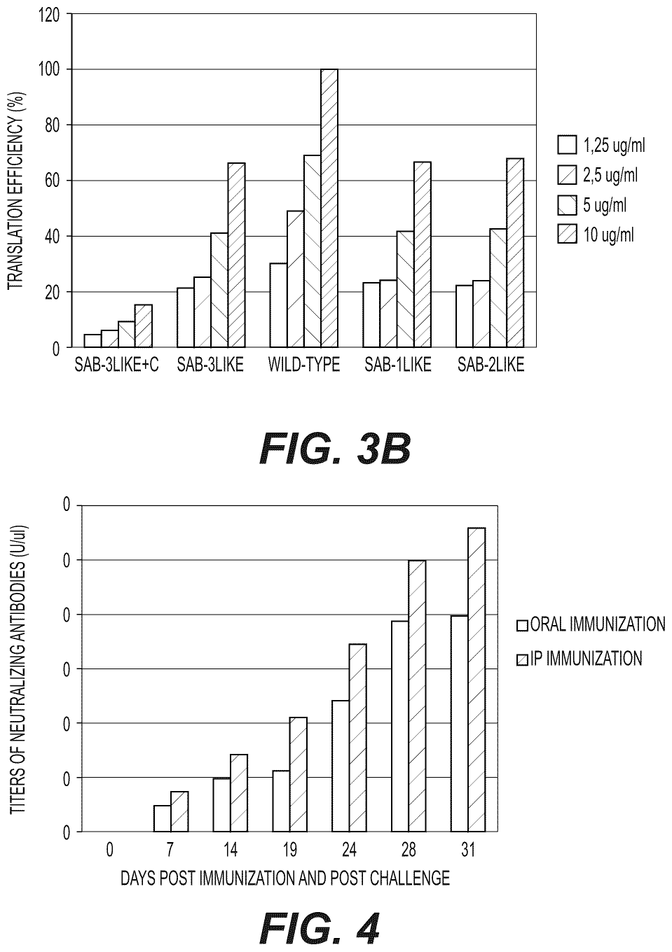

A decrease of protein translation efficiency within the double mutant (SEQ ID NO: 1) was demonstrated in vitro. FIG. 3A is an autoradiograph of the dried SDS-polyacrylamide gel of the RRL-based translation reactions programmed with 20% HeLa cell extracts supplemented with decreasing concentrations (from 25 to 10 μg/ml) of uncapped in vitro transcripts RNA (from left to right). The position of the authentic CVB3 coding region-derived translation polyprotein product is indicated by the arrow. FIG. 3B depicts results of densitometric quantifications of polyprotein products within the Wild-type and mutant strains (SEQ ID NO: 1) in polyacrylamide gel.

### Example 4

### Post-Immunization and Post-Challenge Neutralizing Activity in Mice Sera

Swiss mice were immunized orally (per os) with a boost regimen of CVB3 Live-attenuated containing 100 μl of liquid (˜2 drops approximatively, when using pipette or dropper) of CVB3 Live-attenuated vaccine strain (SEQ ID NO: 1) with a titer of 4.2×10⁵ TCID₅₀ on HeLa cell culture. The liquid suspension used for oral administration included Phosphate-Buffered Saline (PBS) (pH 7.4). Thus, the exact dose used was 4.2×10⁴ infectious particles suspended in 100 μl of PBS.

Post-immunization and post-challenge neutralizing activity induced by the double attenuated mutant against Wild-type CVB3 infection was studied by immunizing mice with the live-attenuated double mutant (Sab-3like+C) (SEQ ID NO: 1). Sera were collected from day 0 to day 14 after immunization and from day 19 to day 31 after challenge, and serially diluted 1:10 to determine the highest dilution able to completely inhibit cytopathic effect by CVB3 wild-type.

FIG. 4 depicts evolution of anti-CVB3 neutralizing antibody after immunization by the live-attenuated double mutant (Sab-3like+C) (SEQ ID NO: 1) at day 0 and challenged by CVB3 Wild-type at day 15.

### Example 5

### Design of Immunization Schedule

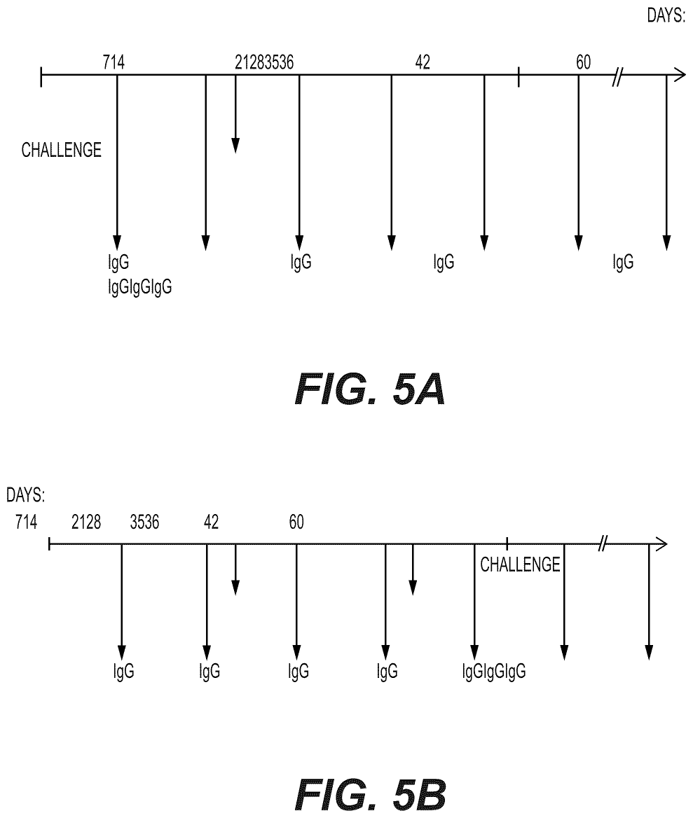

FIG. 5A depicts intraperitoneal (IP) and FIG. 5B depicts Oral (per os) immunization schedule of mice. Mice were immunized intraperitoneally (IP) with a boost regimen at days 0, 15, and 29. Mice were immunized orally (per os) only twice with a boost regimen at days 0 and 15. Neutralizing antibody levels were measured in serum collected from mice at days 7, 14, 21, 28, 35, 42 and 60. Mice were then challenged intraperitoneally (IP) and orally (per os) at day 36 with the wild-type CVB3 Nancy virus strain (5×10⁵TCID₅₀).

### Example 6

Immunization with Mutant or Wild-type Viruses

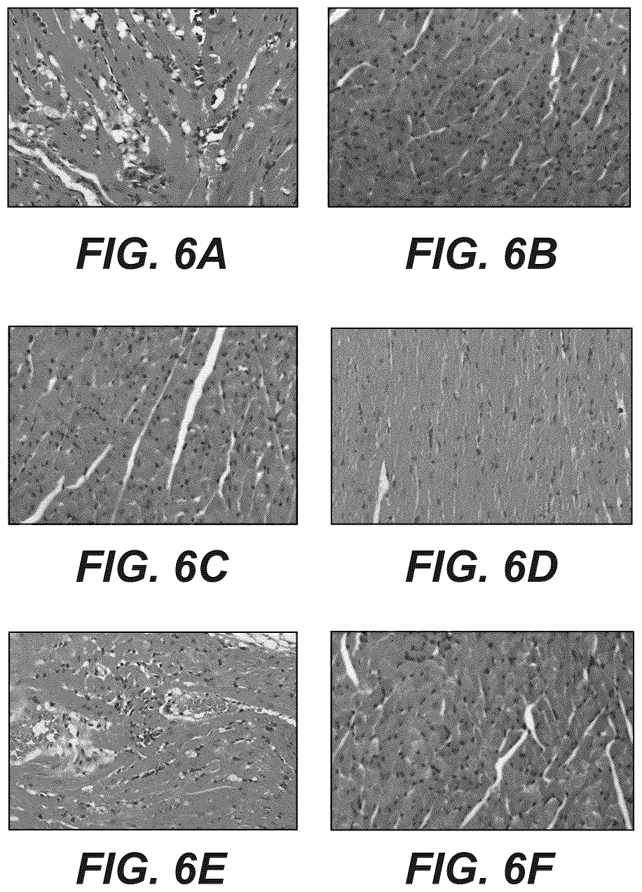

The histology of the hearts of the Swiss mice at day 10 after oral immunization with mutant or wild-type viruses were examined. FIGS. 6A-6F show murine myocardium stained with hematoxylin and eosin, in FIG. 6A from a mouse inoculated with Wild-type, FIG. 6B from a control uninfected mouse, FIG. 6C from a mouse inoculated with Sab-3like mutant, FIG. 6D from a mouse inoculated with Sab-3like+C mutant (SEQ ID NO: 1), FIG. 6E from a mouse inoculated with Sab-2like mutant, and FIG. 6F from a mouse inoculated with Sab-1like mutant (Bars, 200 μm).

Protective immune responses of the two types of immunization routes were as follows. Neutralizing antibodies production after immunization and challenge revealed that the oral route (per os) is stronger than the intraperitoneal route (IP). Results of neutralizing antibodies production in mice sera demonstrated that a second orally (per os) boost regimen is enough to produce the same titers of neutralizing antibodies produced by the intraperitoneal route with three times boost regimen.

In addition, results showed that neutralizing antibodies produced by oral immunization were more sustainable in time post challenge and post immunization than antibodies produced by intraperitoneal immunization. It could be justified by the importance of the prime replication of the attenuated virus in intestine sites when it is inoculated orally.

### Example 7

### Neutralizing Antibody Responses

Mice were immunized intraperitoneally three times at days 0, 15 and 29 with the live-attenuated mutant of CVB3. Mice were only twice_immunized orally (per os) at days 0 and 15 with the same dose of the live-attenuated mutant of CVB3 (SEQ ID NO: 1). All mice were challenged by the wild-type Nancy CVB3 strain at day 36. Control mice received PBS.

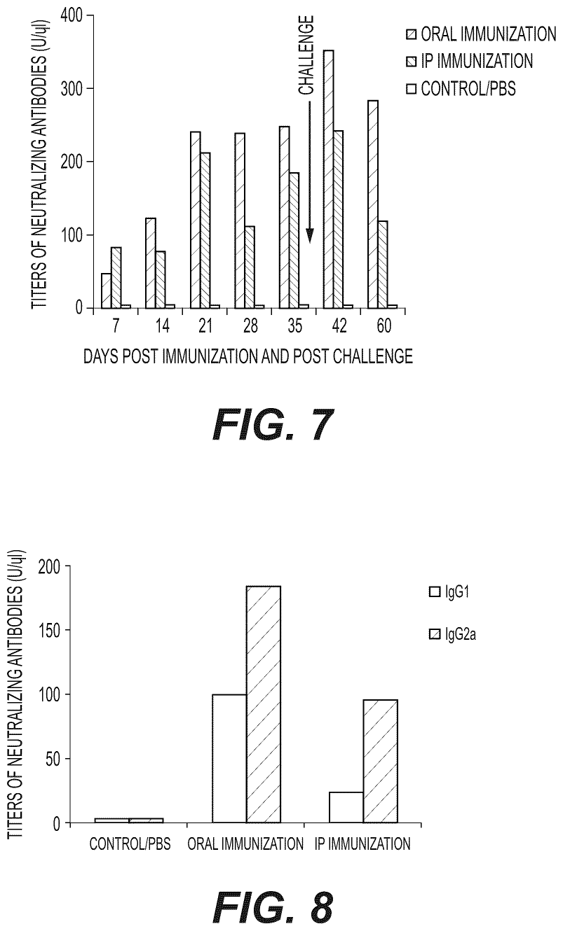

FIG. 7 depicts evolution of neutralizing IgG in sera of mice intraperitoneally (IP) and orally (per os) immunized by the live-attenuated mutant. Neutralizing antibodies production after immunization and challenge revealed that the oral route (per os) is stronger than the intraperitoneal route (IP). Results of neutralizing antibodies production in mice sera demonstrated that a second orally (per os) boost regimen is enough to produce the same titers of neutralizing antibodies produced by the intraperitoneal route with three times boost regimen.

In addition, results showed that neutralizing antibodies produced by oral immunization were more sustainable in time post challenge and post immunization than antibodies produced by intraperitoneal immunization. It could be justified by the importance of the prime replication of the attenuated virus in intestine sites when it is inoculated orally.

### Example 8

### Isotypes of IgG Neutralizing Antibodies

Isotypes of IgG, IgG1 and IgG2a neutralizing antibodies in the serum of intraperitoneally and orally immunized mice were evaluated at day 60. Control mice received PBS. As shown in FIG. 8, serum IgG1 and IgG2a recognized CVB3 viral protein VP1. Total IgG levels and subclass titers showed strong correlation with the immunization route. IgG1 antibodies were elevated in the sera of mice twice immunized by the oral route. The mice group orally immunized by the live-attenuated strain (SEQ ID NO: 1) and challenged by the wild-type strain showed increased levels of Th1 (Lymphocytes T helper) related subclass IgG2a compared to mice three times intraperitoneally immunized and challenged with the wild-type in the same conditions. Control/PBS mice illustrated very low titers compared to all other groups.

### Example 9

### Body Weight Changes Observed Intraperitoneally (IP) and Orally (Per Os)

FIG. 9 shows body weight changes observed in intraperitoneally (IP) and orally (per os) immunized mice seven days after challenge by the wild-type Nancy CVB3 strain. The control challenged mice group showed a substantial decrease in body weight percentage. Decrease in body weight is an indicator of viral infection. Following challenge, all control challenged mice showed more than 25% loss in body weight. There was also a significant decrease in body weight (20%) within the intraperitoneally immunized group. All other groups showed slight fluctuations in body weight. However, no significant loss in body weight was observed within the control/PBS mice and control/not challenged mice.

### Example 10

### Lethality of Mice as Indication of Pathogenic Virulence Infection.

After 14 days post challenge by the wild-type CVB3 Nancy strain, all control challenged mice showed complete lethality of the group (100% mortality). There was also a significant decrease of survival mice (25%) intraperitoneally (IP) immunized. All other groups of control/PBS not challenged and orally (per os) immunized showed no mortality. Percent of survival intraperitoneally (IP) and orally (per os) immunized mice after a challenge with wild-type CVB3 Nancy strain in comparison to that of the negative control (Control/PBS). FIG. 10 shows percent of survival intraperitoneally (IP) and orally (per os) immunized mice after a challenge with wild-type CVB3 Nancy strain in comparison to that of the negative control (Control/PBS).

### Example 11

### Decrease of Replication of the Wild-Type Virus

FIG. 11 shows titers calculation on HeLa cell culture of virus isolated seven days post challenge from mice organs. HeLa cells were plated at a density of 1×10⁶ cells. Intestine and heart homogenates were collected from all immunized groups, used as viral stock samples, and added to the cells. The viral titers are expressed as tissue cell infectious dose/ml (TCID₅₀). Control mice received only PBS, whereas control challenged received only wild-type dose at challenge day 36.

A decrease of replication of the wild-type virus in organs of orally (per os) challenged mice group was noted. Viral titer of wild-type virus was shown to be 30-fold lower in heart and intestine of orally (per os) vaccinated mice compared to intraperitoneally (IP) vaccinated mice. As expected, the viral load in the control challenged mice group was considerably high.

### Example 12

Histology of the Hearts of Mice Seven Days after Challenge

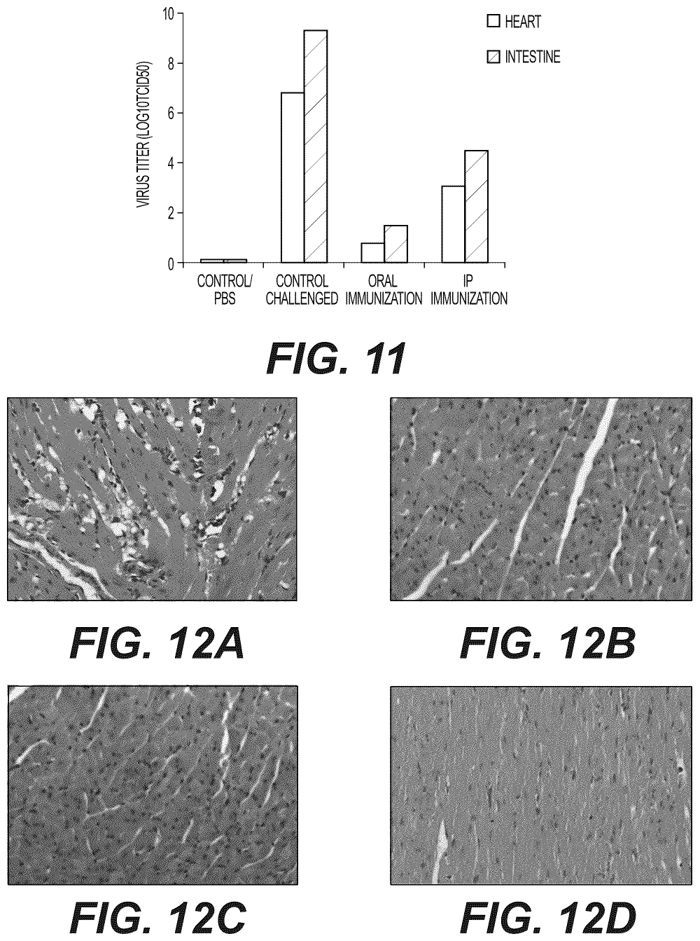

Mice were three times intraperitoneally (IP) and only twice orally (per os) immunized with the live-attenuated CVB3 strain (SEQ ID NO: 1), then challenged by the wild-type CVB3 Nancy strain. FIGS. 12A-12D show myocardium tissues stained with haematoxylin and eosin from (FIG. 12A) control challenged mice; (FIG. 12B) from twice orally immunized, then challenged mice (FIG. 12C) mice three times intraperitoneally immunized, then challenged (FIG. 12D) and from control non challenged mice (Bars, 200 μm).

Oral (per os) immunization by the live-attenuated CVB3 strain (SEQ ID NO: 1) exhibits a more attenuated phenotype in mice groups than with the intraperitoneally (IP) route. The microscopic examination of heart tissues showed no inflammatory damages in mice orally vaccinated. Tissues appeared normal and indistinguishable from heart from mock-infected mice. As expected, the wild-type Nancy CVB3 strain provoked widespread inflammatory lesions and significant extension of infiltration and necrosis area (myocarditis) in heart muscles of control mice challenged.

It is to be understood that the Coxsackievirus B3 vaccine is_not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A composition, comprising:
a live, attenuated double mutant strain of Coxsackievirus B3 comprising SEQ ID NO: 1, wherein cytosine is substituted by uracil at position nt. 473 and uracil is substituted by cytosine at position nt.475 in the genome of the wild type Coxsackievirus B3 in domain V within the internal ribosome entry segment (IRES), wherein
the composition is formulated as a liquid suspension for oral administration, and
the liquid suspension is provided within a capsule, the capsule being non-gastro-resistant.

2. (canceled)

3. The composition as recited in claim 1, wherein the liquid suspension comprises Phosphate-Buffered Saline.

4. (canceled)

5. A method of vaccinating a subject against a Coksackievirus B3 (CVB3) strain infection, comprising administering to the subject the virus-like particle (VLP)-based vaccine as recited in claim 1 in an amount effective to elicit an immune response directed against the CVB3.

6. A method of vaccinating a subject against a Coksackievirus B3 (CVB3) strain infection, comprising administering to the subject vaccine against Coksackievirus B3 (CVB3) including a live, attenuated double mutant strain of Coxsackievirus B3 (SEQ ID NO: 1), wherein cytosine is substituted by uracil at position nt. 473 and uracil is substituted by cytosine at position nt.475 in the genome of the wild type Coxsackievirus B3 in domain V within the internal ribosome entry segment (IRES).

7. The method as recited in claim 6, wherein the vaccine is administered intraperitoneally or orally.

8. The method as recited in claim 6, wherein the vaccine is formulated as a liquid suspension for oral administration.

9. The method as recited in claim 8, wherein the liquid suspension comprises Phosphate-Buffered Saline.

10. The method as recited in claim 8, wherein the liquid suspension is provided within a capsule.
